(19) United States
(12) Patent Application Publication
(10) Pub. No.: US 2008/0279995 A1
(43) Pub. Date: Nov. 13, 2008

(54) Process for the More Effective and Gentle Release of Quality-Enhancing Constituents From Grapes, the Young Wine Obtained in the Process and the Wine Produced Therefrom, as Well as a Device for Carrying Out the Electroporation

(75) Inventors: Christoph Schultheiss, Pfinztal (DE); Rolf Nuesch, Karlsruhe (DE); Hanns-Gunther Mayer, Karlsruhe (DE); Jurgen Sigler, Freiburg (DE); Martin Kern, Waghausel (DE)

Correspondence Address: VENABLE LLP, P.O. BOX 34385, WASHINGTON, DC, 20043-9998, US

(73) Assignee: Forschungszentrum karlsruhe GmbH, Karlsruhe (DE)

(21) Appl. No.: 10/593,694

(22) PCT Filed: Mar. 4, 2005
(86) PCT No.: PCT/EP2005/002282

§ 371 Date: Jul. 25, 2008

(30) Foreign Application Priority Data
Mar. 20, 2004 (DE) 10 2004 013 762.5

(51) Int. Cl. C12G 1/02; A23N 1/00; C12M 1/42

(52) U.S. Cl. 426/238; 426/592; 435/173.6; 435/285.2

(57) ABSTRACT

An improved and more gentle process for extracting useful substances from wine grapes is achieved by electroporation of the must produced from red and/or white grapes. The must is pumped or circulated before the pressing applied for must production through an installation or part of an installation and pulsed electric fields are applied therein to irreversibly rupture the biological cell walls of the grape skins. The device for carrying out must electroporation comprises a dielectric pipe, the flow duct for the must, in whose wall are arranged two mutually spaced electrodes to form a pulsed electric field between the electrodes. The must, which forms the electrolytic load of the device, has an electric resistance at least as high as the impedance of a high-voltage pulse generator connected to the device.

The invention relates to a process for an improved and gentler release of quality-enhancing constituents from grapes, a young wine produced with said process, and the wine produced therefrom, as well as a device for carrying out the electroporation process.

The term electroporation refers to the opening up of cells by subjecting them to an electrical field, meaning the pore-shaped opening of the wall of a biological cell. This pore-shaped opening of a cell wall is reversible when caused by a weaker electrical field, meaning such a pore closes again once the field is no longer active, but is irreversible if stronger electrical fields are activated. That is to say, such a pore remains open/torn up even after the field is deactivated. The electroporation process was first introduced with German Patent 123 741, in the year 1960. The term electroporation by itself refers to a non-thermal opening of the cell walls, which is designed to achieve a gentle release of the quality-enhancing cell constituents.

The process of electroporation for breaking up biological cells is of interest to the food industry, on the one hand because of the gentle breakup of the cells and, on the other hand, because the electroporation is an energy-saving process step. With traditional opening up/breaking up processes, the plant material to be processed is heated with thermal and thus very expensive energy, so that the cell walls become permeable once a sufficient amount of thermal energy has been applied. However, many constituents that are important from a nutritional-physiological point of view are damaged or decompose during the thermal process and/or undesirable constituents are mobilized.

With other traditionally used processes, the plant material to be processed is mechanically crushed to a pulp, which makes the following pressing-out operation more difficult and results in a pressed cake with low concentration of solids and/or results in a high loss of fruit juice/young wine and the like.

Increasingly, enzymes are also used for opening up the cell membranes. However, these have the disadvantage of restricted licensing for use and high expense, of changing the taste of the fruit juice/young wine, and of resulting in imperfect or poor quality fruit juice or wine produced therewith.

Ethyl alcohol is used in many cases for extracting the constituents. The alcohol dissolves the cell walls, which are composed of fat molecules, thereby releasing the quality-enhancing constituents.

All processes for extracting/breaking-up biological cells are primarily evaluated on the basis of harmlessness of the products obtained therewith and from an economic point of view by the cost associated with the process.

In the fruit juice/young wine production, extremely ingenious processes are used for extracting the biological cell substances. In particular, this is true for the wine production, where the individual processes for creating white wine and red wine differ considerably. The further processing into the different types of wines depends on the substances contained in the young wines, which form the basis for the quality which can be obtained.

For the traditional wine production, the harvested grapes are removed from the stems, de-stemmed in expert jargon, and are then mashed into a must for obtaining the young wine. Depending on the demanded wine quality, this is followed by a 10-day must fermentation period, for example, which frequently takes place in special double-walled containers that are heated to temperatures of up to 38°, where the alcohol fermentation starts. The alcohol content, which increases in concentration, is used for dissolving the cell membranes in the grape skin. Cell constituents such as pigments, tannins, aromatic agents, as well as other water-soluble and alcohol-soluble substances and plant-specific proteins are extracted in this way. The extraction of the tannins is slowest and is generally not complete after 10 days.

The must-fermentation process is primarily used for producing high-quality red wines since correspondingly high-quality grapes are available for this. However, the heating and storage costs for this process represent an important cost factor that cannot be neglected. If the red grapes are of a lower quality or intended for mass-produced red wines, the must-heating process is frequently used. With this process, the must is heated briefly with the aid of heat exchangers, for example to 85° C. for a period of 1 minute. An orientation value of approximately 10 liters of heating oil for each ton of must is used for generating steam. To be sure, the heating of the must produces a good pigment yield, but results in particular in the lower extraction of tannins because of the watery-thermal extraction conditions. Rather smooth wines with little depth and longevity are therefore produced with this process, which are intended for early consumption and cannot be stored for extended periods of time, wherein the appearance of a cooking aroma can be noticed at times.

White grapes are generally pressed out immediately after the grapes are mashed into a must, if applicable following a brief residence time, so that the constituents in the grape skin do not enter the young wine to a high degree, but remain in the pomace. With wines having a so-called bouquet, however, a pre-crushing of the grape skin cells is necessary for extracting the aromatic substances and their preliminary stages. As a rule, this is achieved by having a longer must residence time, wherein natural enzymes that are characteristic for grapes and/or added enzyme preparations cause the breakup. Longer must residence times of this type are generally also used for white wines with higher emphasis on tannins, as well as for a better extraction of the nitrogen substances etc. needed for the fermentation. However, this process carries the risks that undesirable enzymes/proteins become active and that damaging micro-organisms and yeasts will grow.

It is the object of the present invention to develop a process for a faster, better, and gentler release of quality-enhancing constituents from grapes, wherein this is reflected in the quality of the young wine obtained from the must and, in turn, also in the wine produced therewith. Furthermore proposed is a device for the effective and energy-saving realization of the irreversible electroporation of grape skin cells.

This object is solved with the process steps disclosed in claim 1. Claim 6 discloses the result of this process, the fruit juice/young wine produced from the at least partially electroporated must, as well as the wine produced therewith. The grape must can be processed with a device as disclosed in claim 7 for generating a pulsed, electrical field with a strongly inhomogeneous field distribution in the flow volume, at least in some sections, through which the must flows either continuously or in batches for the irreversible electroporation.

The process for obtaining an improved and gentler release of the quality-enhancing constituents from grapes involves the following steps: The must produced from the red and/or white grapes is heated to a temperature, or maintained at a temperature, which is at least above the freezing temperature for the must and which can be adjusted up to the traditional temperatures known for heating a must. Prior to the pressing operation for obtaining the young wine, the must is pumped/flows through a device where it is subjected to pulsed electrical fields, extending across the flow channel cross section, to achieve an irreversible opening of the walls of the biological grape skin cells, meaning the process of electroporation. The pulse duration is in the range of 0.5-3 μs. The must is subjected at least once and preferably several times to the pulsed electrical field, with such high electrical field intensities that on the one hand the difference in the potential at the grape-skin cells measuring 7 to 10 μm is at least 100V and is sufficient for the irreversible electroporation while, on the other hand, the streamer formation limit of approximately 1000 kV/cm is not reached. It has turned out that the specific dose of energy required for the electroporation of cells is between 10 and 40 kJ per kilogram of must, for a must temperature ranging from 10° C. to approximately 40° C. Below a temperature of approximately 10° C., it should be adjusted upward by a factor of 2 to 4 and for temperatures above approximately 40° C., it should be adjusted downward by a factor of 2.

The electroporated must is then subjected to a predetermined residence time to achieve a rapid, gentle, and energy-optimized release of the quality-enhancing constituents from the fruit skin. This release is monitored by taking at least one sample from the must and by recording the data obtained for the substances and their respective concentrations. Following the residence time, the electroporated must is finally pressed out for obtaining the young wine, which forms the basis for producing the actual wine.

Additional processing steps are provided in the dependent claims 2 to 5. According to claim 2, the must of white grapes is thus subjected to a residence time ranging from a few minutes to several hours following the electroporation.

In contrast, claim 3 discloses that the must obtained from red grapes is subjected to a residence time ranging from a few hours to several days following the electroporation process.
From this it follows that for producing the young wine, the electroporated must only needs to be subjected to a press-out pressure that equals the one used in the traditional young wine production, wherein experience has shown that a 30% lower pressure is quite sufficient.

The must is moved either continuously or in batches through the device for electroporation to achieve the irreversible electroporation, wherein the continuous flow is normally used.

Starting with traditional systems for the wine/young wine production in wine cellars, the equipment for carrying out the step of irreversible electroporation of the must, as disclosed in claim 7, comprises a dielectric pipe functioning as must flow channel, with a simple round or simple polygonal, but at least four-cornered cross section. With a round pipe cross-section, the contour line should be curved continuously toward the outside, curved continuously or changeable, meaning there should be no change in direction of the curvature radius. With the polygonal cross section, immediately adjacent jacket segment surfaces should form an angle of ≧90° toward the inside and the cross section should also be curved only toward the outside. The most favorable flow conditions for the must are thus obtained for the round and polygonal cross sections. At least two spaced-apart electrodes are installed in the wall of this flow channel. The electrodes are positioned countersunk or flush with the wall of the flow channel or they project from this wall into the flow channel.

The total surface area of the blank electrode surfaces projecting into the flow channel determines the control/delimitation of the current between the electrodes.
The clear cross section and the length of the flow channel for the device are designed such that the must as electrolytic load of the device is at least equal to the impedance of a high-voltage pulse generator that is connected to the device. As a result, the boundaries for dimensioning the flow channel and the exposed electrode area are predetermined.

Claims 8 to 11 contain different specifications for the device used for the electroporation of the must.

The respective frontal surfaces of the electrodes, which are exposed to the flow channel and function to generate the pulsed electrical field between the electrodes, are positioned opposite each other and perpendicular to the flow axis, as shown in claim 8. They are either lines up as electrode pairs in flow-axial direction, spaced apart at the required spacing, or in a zigzag line oriented in axial flow direction, or spirally wound around the flow axis.
According to claim 9, the electrodes are ring-shaped and are lined up successively, spaced apart at the required spacing, in coaxial direction to the flow axis.
According to claim 10, the electrodes are pin-shaped, they project radially into the flow channel, and are positioned as described in claim 8.
To avoid excessive increases in the electrical fields, the electrodes have rounded contours, as described in claim 11. The electrical field intensity should not exceed 1000 kV/cm to avoid the danger of streamer formation, which results in a strong and very disruptive chemical decomposition of the must.

As specified in claim 12 for the type of building material required for the flow channel according to the food laws, the flow channel and therein installed electrodes must be composed of a material that is inert to the process and is suitable for use with food items, or a material of this type should at least cover the surfaces which come in contact with the must. PE [polyethylene], for example, is a suitable dielectric material while stainless steel meets the requirements for the electrode material.

The mass flow rate per time unit determines the dimensions of the clear surface of this electroporation device, as well as the flow speed, wherein no backup should occur during the operation. Obstructions that may result in such backups should be avoided, which is another reason why the electrodes have rounded contours.

The irreversible electroporation of grape cell walls in the must refers to a process for producing young wine, for which the plant cells are opened up gently by means of the pulsed electrical field and the important, quality-enhancing constituents are effectively released. The irreversible electroporation has the advantage of a fast, in particular non-thermal extraction of pigments, tannins, aromatic agents, and the other important constituents needed for the wine production, such as enzymes, wherein these also include nitrogen substances from the grape-specific proteins.

For the wine-producing industry, the electroporation of cells represents an energy-saving, economic alternative to the must fermentation, meaning the fermentation following a heating of the must, and/or to longer residence times for the must. The electroporation of the must from grapes makes it possible to optimize the machine capacities and, in the final analysis, leads to an at least comparable quality in the wine production. The irreversible and thus effective cell breakup furthermore leads to a better extraction of the nitrogen substances, for example supplied by the dissolved-out proteins, which are necessary for the yeast assimilation. This contributes to an improved fermentation and to wines with a long shelf life by avoiding the so-called atypical aging (ATA) characteristic.

To understand the process parameters used, it is necessary to take a closer look at the mechanism of the irreversible electroporation of plant cells, particularly the cells in the grape skin. The inner parts of the biological cells such as the cell core, the cytoplasm, and the like are separated from the outside by the cell wall or cell membrane, which consists of an extremely thin layer on the basis of fat molecules, also called a bilipid layer. One important biological function of the cell wall is its capacity to generate ionic channels with the aid of electrical potentials generated by the cell itself, wherein the natural electrical potentials generally have values below approximately 70 mV. The potential can be generated artificially by means of outer electrical fields and can be raised to a level where the opening of the cell walls becomes irreversible, wherein the potential is determined by multiplying the effective path of the field line in the cell with the value for the field intensity. For example, if the field intensity at the cell location is 10 kV/cm and if the cell at the location of the field line has a diameter of 10 μm, then the potential is computed to be 10 volt. With respect to this, literature (e.g. see: K. H. Schoenbach et al., “Bacterial Decontamination of Liquids with Pulsed Electric Fields,” Transactions on Dielectrics and Electrical Insulation, Vol. 7, No. 5, pages 637-654, October 2000) provides the following information: in the case of long pulses in the ms range, the potentials must be on the order of magnitude of 1 volt. For shorter pulses, meaning pulses in the μs range, the potentials at the cell membranes must be raised to values of up to 10 volt.

Experiments have long been carried out and models set up for demonstrating the mechanism of cell poration with the aid of bacteria. One important result of these experiments relates to the dynamic of cell poration. Whereas following the generating of an electric field the polarization effects of the salts contained in the cytoplasm are recorded on a 100 ns scale, the dynamic of the irreversible opening of pores in the cell membrane requires time with a higher order of magnitude. The analyses show that the process becomes irreversible only if the pores have opened up unnaturally wide, wherein this requires approximately 1 to 3 microseconds, with a cell poration potential of 10 volt. It means that to achieve a potential of at least 10 V for each cell, electrical field intensities of at least 14 kV/cm are required for a grape skin having an average cell diameter of 7 μm. However, experiments have shown that the grape skin cell poration is not complete with these field intensities and that the pigment outflow (anthocyans) is incomplete even after a period of 24 hours. A potential of at least 100V is therefore required for a secure electroporation. Electrical field intensities of at least 140 kV/cm are required for this, which can only be generated locally with the aid of an inhomogeneous field configuration.

It is critical for the realization of this process that the energy used for the electroporation of cells is supplied in a pulsed manner. For a grape must, the conductivity of the suspension is around 0.26 S/m. For example, if an electrical field with the intensity of 10 kV/cm were applied to a cube-shaped dielectric trough filled with grape must, which has a side length of 10 cm and two electrodes installed on opposite sides of the wall, this would result in a current flow of approximately 20 kA, which would lead to a power consumption of approximately 2000 MW, thereby corresponding to the power output of a power station. It is therefore unrealistic to want to carry out the electroporation of cells with processes using switched direct and alternating current. Rather, such peak capacities can only be generated with high-power pulsed systems.

The pore-opening dynamic is highly dependent on the temperature. The cause lies with the thermal fluctuations of the lipid molecules in the bilipid layer of the cell membrane/skin. Statistically, pores with a diameter of approx. 1 nm form under the effect of the temperature, which then close again quickly. It is critical that for the duration of the pulse, one or several pores are opened wide enough under the effect of the sufficiently high electrical field, so that the opening is irreversible. The colder the material to be processed, the more intensive the electroporation of cells must be with respect to field intensity and energy. In numbers, it means that the values for the energy dose would have to be higher by a factor of 2 if the temperature of the must is below 10° C. In the same way, the values for the energy dose can be lower by a factor of 2 if the must temperature is 30 to 40° C.

The irreversible electroporation is explained in further detail in the following, showing in:

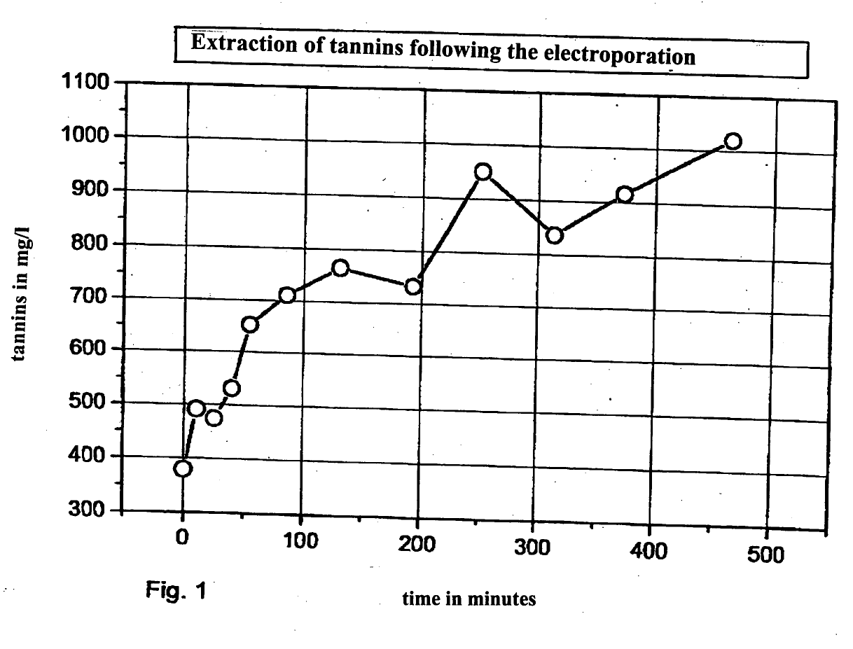

FIG. 1 the chronological course of the tannin extraction;

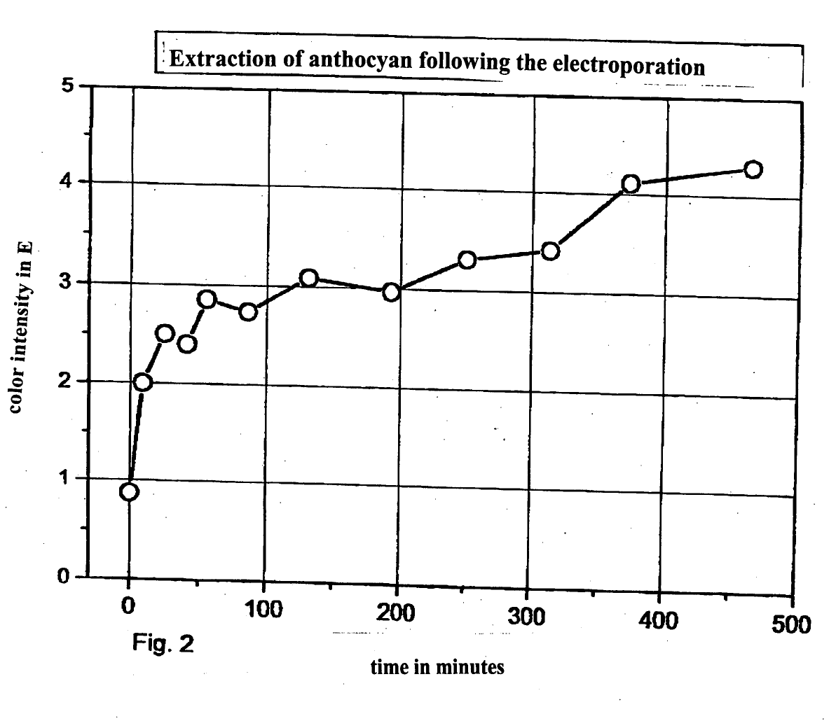

FIG. 2 the chronological course of the pigment extraction;

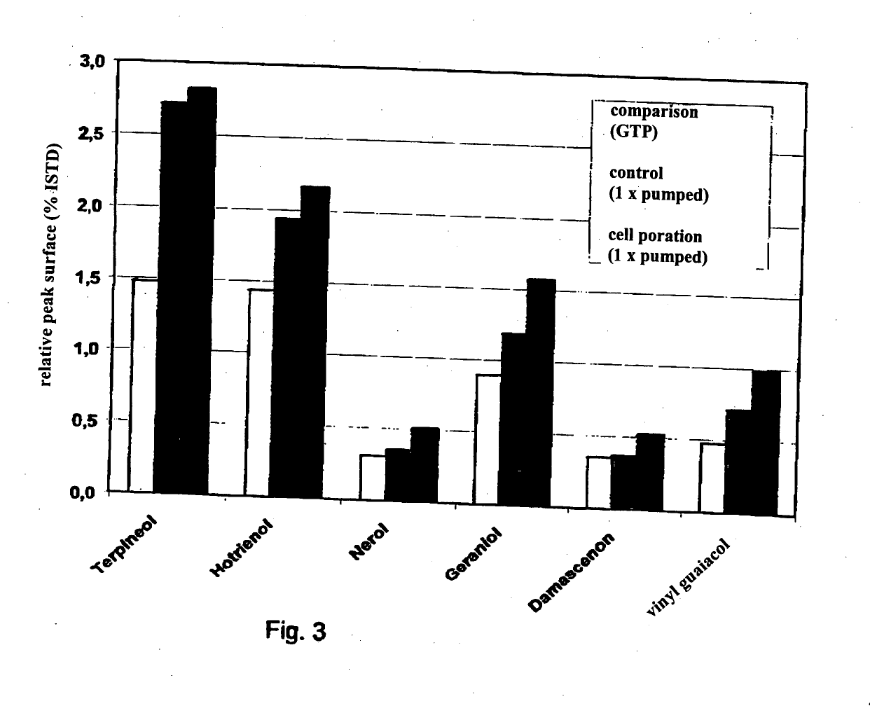

FIG. 3 aromatic agents of riesling wine following the cell poration of the must;

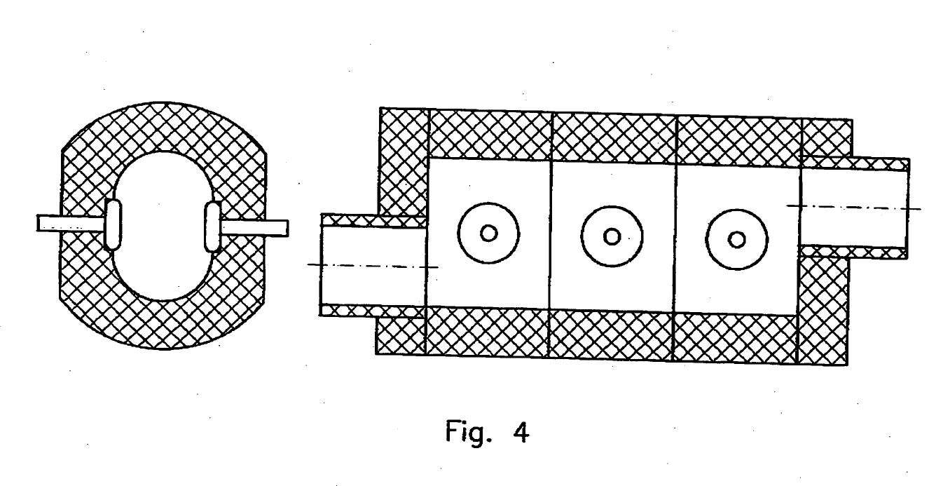

FIG. 4 opposite-arranged electrodes;

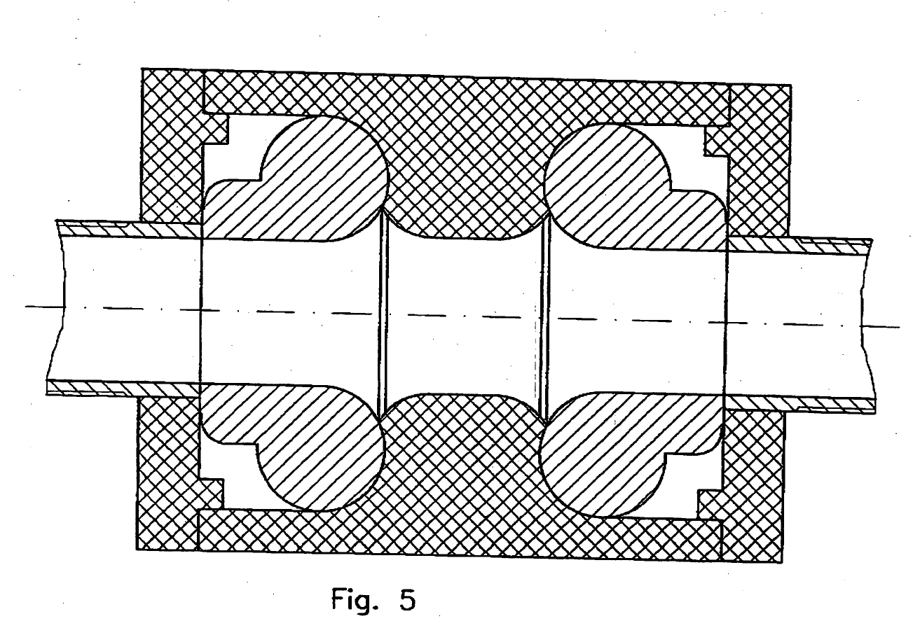

FIG. 5 coaxial, opposite-arranged electrodes;

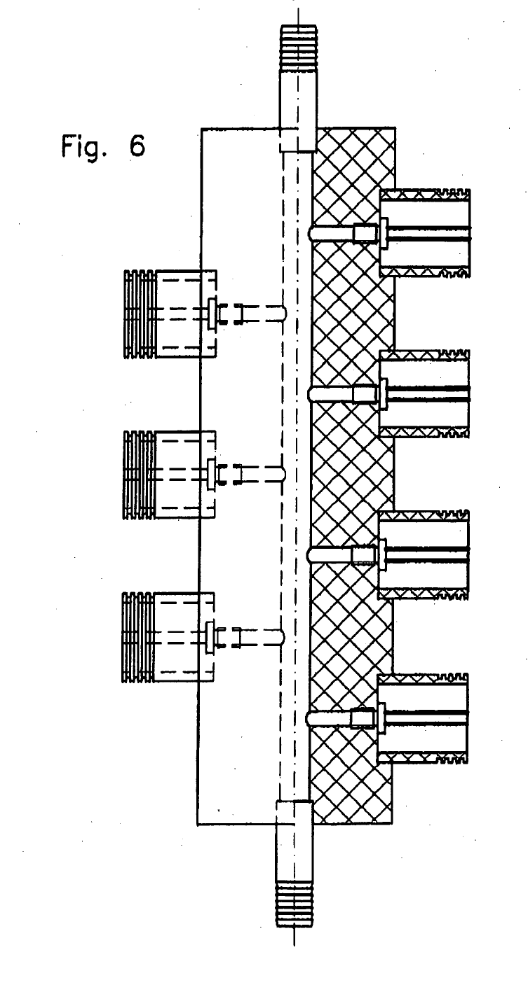

FIG. 6 an arrangement of rod-shaped electrodes;

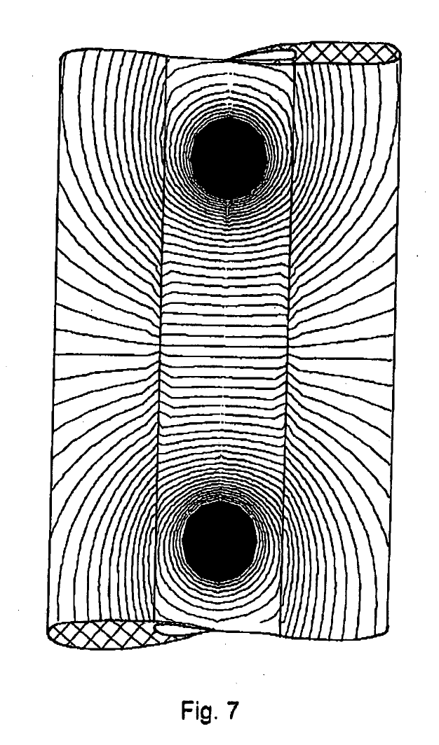

FIG. 7 inhomogeneous potential-line distribution between two rod-shaped electrodes.

### RED WINE PRODUCTION

The must for a late-vintage burgundy is pumped at room temperature with the aid of a food pump through a pipe system for a reactor arrangement with inhomogeneous field (see FIG. 6). The pumping capacity is 1000 l/h and the repetition frequency for the 300 kV pulses is 10 Hz, corresponding to the specific energy of a 20 kJ/kg must. Alternatively, the temperature of the red grape must inside a reactor with nearly homogeneous electrical field (such as the one shown in FIGS. 4 and 5) is raised to 30 to 40° C. The control product is produced by heating the must, in accordance with the traditional process for breaking up grapes. It turns out that the chronological course of the extraction process during the electroporation of cells is comparable to that of the thermal denaturation. FIGS. 1 and 2 show the chronological development of tannins and the pigment intensity of red-grape must, following an irreversible electroporation of cells. The extraction behavior is comparable to that of heating the must, namely that the primary extraction of tannins and pigments occurs during the first two to three hours, with the exception that for this example the electroporation of cells in the must occurred at room temperature.

As shown in Table 1, the young wine from red grapes, produced with the aid of cell poration, has only a slightly lower tannin and acid content than the one obtained by heating the must. However, this can be influenced by varying the parameters. In both cases, this is followed by the steps of pressing-out, pre-clarification, and fermenting. The key values for the table-ready wine in particular also correspond to the pigment and tannin values of the control product.

During a blind taste test conducted with 48 competent experts in the field of viticulture, both variants proved to be equally good, wherein the product obtained by cell electroporation was ranked number 1 in 23 cases and the control product in 25 cases. An evaluation based on the 5-point checklist of the German wine regulations showed that the red wine produced with the aid of irreversible cell electroporation on the average reached the quality grade of 2.15 while the control product reached the quality grade 2.17, wherein n=42 events, which is considered non-distinguishable in this case. The example shows that the electroporation of cells delivers results that are at least comparable to those obtained by heating the must.

White Wine:

The electroporation provides a good starting basis for the production of white wine, as shown with the following example. The harvested riesling grapes were destemmed, then crushed into a must, which was subsequently pumped through the device with the electroporation turned off (for control), as well as with the cell electroporation turned on. The mechanical strain of the must therefore was the same and any possible differences must be ascribed solely to the additional effect of the electrical fields. For a further comparison, harvested grapes of the same type were processed by means of whole-grape pressing or GTP.

As expected, the whole grape pressing (GTP) resulted in the lowest pomace content for the young wines that were produced. The pomace content of the variant produced with electroporation of cells is higher, which is mostly due to the pumping load.

There are only insignificant differences in the pomace content of young wines pre-clarified of sedimentation. With this example, the variant obtained by electroporation of cells differs noticeably in that it has a considerably lower acid content on the one hand and a higher content in tannin and yeast-available nitrogen (fermentation N-value) on the other hand. These are advantages in view of avoiding the atypical aging (ATA) characteristic.

Wine ready for consumption which is produced with the aid of electroporation has a higher tanning content as well as a higher sugar-free extract. The clearly increased potassium value of this variant indicates an extremely effective cell breakup.

Of interest to the white wine production is the release of aromatic substances or their preliminary stages, especially from grape skins. FIG. 2 shows that the whole-grape pressing (GTP) results in the lowest contents of terpenes and other aromatic substances. The release of aromatic substances is improved by musts in connection with a pumping operation, wherein the additional electroporation in this case resulted in a further noticeable increase. The electroporation promotes the dissolving out of low-molecular nitrogen substances, amino acids, and ammonium from the cell. This is furthermore desirable since the proteins represent nitrogen carriers for the yeast assimilation and because an insufficient action of these results in faulty fermentation and an atypical aging characteristic ATAC.

The result of a sensory evaluation is shown in the following:

A panel of 50 expert vintners analyzed the comparison variant obtained with the process of whole-grape pressing to determine its tendency to develop an atypical aging characteristic (compare low fermentation N-value of new wine in Table 2). The same analysis was made for the somewhat crude control variant; obtained by creating a must and using 1 pumping operation. The completely extracted variant that is opened up by means of additional electroporation was clearly preferred and was ranked number 1 with a significant lead. Table 2 contains further details of the test result.

Advantages of the cell poration therefore exist for the white wine production and include a better extraction of the grade-specific aromatic substances and preliminary aroma stages, as well as avoiding of the atypical aging characteristic (ATAC).

However, a strong cell break-up such as for the red grape must is not desired for the white-grape must because the associated dissolving out of the tannins changes/influences the character of the white wine. The electrical field intensity and specific energy can be reduced. FIGS. 4 and 5 show reactor concepts with larger flow cross sections and a field distribution with lower electrical field intensity, which extends over a larger volume. In FIG. 5, the electrodes are arranged axial symmetrical while in FIG. 4, they are arranged radial. With this electrode configuration, large-volume electrical fields can be generated, which are nearly homogeneous in the region of the field axis. In both cases, the inhomogeneity of the electrical field is reduced in favor of a more homogeneous field over a larger volume area by selecting planar electrodes and large curvature radii for the electrodes. In both cases, the mean electrical field intensity for 300 kV pulses amounts to approx. 60 kV/cm in the amplitude.

With all reactor configurations, at least two electrodes are installed in the dielectric pipe, which subject the flow of must at least once to a pulsed high-intensity field in the flow volume. The exposed surface area of the electrode surfaces projecting into the flow channel is used for the control/delimitation of the flow between electrodes, wherein the electrolytic load of the device for the irreversible electroporation is at least approximately adapted to the impedance of the connected high-voltage pulse generator. The load is advantageously higher as compared to the generator impedance since this allows higher currents to flow through the must. The currents locally generate in the must the necessary electrical fields for the electroporation of cells. However, higher currents of, for example of 20 kA, reduce the life expectancy of the high-voltage generators used.

FIG. 6 shows a reactor version used for the irreversible electroporation of red-grape must. This version consists of a dielectric pipe, meaning the flow channel for the must, which has a round or polygonal, but at least a four-cornered cross section with a surface corresponding to approximately 4 cm². At least one pair of electrodes is installed in the pipe wall, spaced apart at a distance of 6 cm, such that the two electrodes are installed flush with the wall and project into the flow channel, wherein the electrode rods have curvature radii in the range of r=6 mm. The electrical field axis in this case intersects at an angle with the flow axis. Shown in FIG. 7 are the electrical potential lines between two rod-shaped electrodes, which generate the electrical field and indicate the strong, distinctive inhomogeneity of the electrode arrangement. In the embodiment according to FIG. 6, the electrical field intensity in the area immediately surrounding the electrode reaches values of up to 230 kV/cm for a 300 kV pulse amplitude. The desired electroporation potentials of >100 V can be generated locally in the area surrounding the electrodes.

With respect to the electrode surface area exposed to the flow channel, all three electrode configurations are based on the following critical dimensions that are important for the electrode surface:

FIG. 4: The three electrode pairs, also of non-rusting steel, are arranged to project in radial direction. The disk-shaped electrodes have a diameter of 40 mm and a curvature radius of 10 mm. The spacing between electrodes is 50 mm, wherein field intensities of 45 to 80 kV/cm will adjust in the pulse amplitude for a pulse of 300 kV, depending on the location. The flow channel has an oval cross section to avoid parasitic electrical discharges via the inside wall. FIG. 5: The electrodes are arranged axial symmetrical and consist of non-rusting steel, stainless steel, while the insulating body is made of polyethylene. The diameter of the flow cross section is 50 mm while the spacing between the rounded electrodes is approximately 70 mm. The curvature radius for the curved surface pointing toward the counter electrode is 20 mm. The maximum occurring field intensity for a 300 kV pulse does not exceed 50 kV/cm. FIG. 6: The flow channel has a diameter of 20 mm. The stainless steel electrodes are spaced apart by 60 mm for realizing the high-voltage resistance¹ and form electrical fields in both directions. Exceptions are the marginal electrodes, which are connected to ground and generate only one field in the direction of the adjacent electrode under high voltage. FIG. 6 ¹Note: Hochspannungsfestigkeit=high-voltage resistance; Hochspannungsfeldstarke=high-voltage field intensity shows fields generated by 6 electrode pairs, formed with 7 electrodes.² The electrodes respectively project semi-spherically into the channel and have a curvature radius of 6 mm. The field intensity for a 300 kV pulse varies between 40 and 230 kV/cm at the apex value. The three above-described embodiments represent examples for dimensioning.² Note: This sentence is not clear in the original.

The electrodes used in the device for the irreversible electroporation are connected to the output of a high-voltage pulse generator, the electrical energy source. The successively following or lined-up electrodes in the flow channel are alternately connected to a reference potential, in most cases the ground potential, and to the high-voltage output of the associated high-voltage pulse generator. With an even number of electrodes, at least one electrode pair or successively lined-up electrode pairs are present while for an odd number of electrodes, the first and last electrode is advantageously connected to the reference potential for reasons of safety. When using a device such as the one shown in FIG. 4, which has a radial electrode arrangement, bipolar output pulses from the pulse generator are useful for insulation-technical reasons. The reference potential adjusts itself automatically between the two electrodes that generate a field.

A Marx generator or a Marx generator embodied as LC ladder network offers itself as generator for generating high-voltage pulses in the microsecond to sub-microsecond range, with an amplitude between 300 and 500 kV and rise times in the range of 100 ns, at current intensities below 10 kA and pulse lengths of about 1 microsecond. A Marx generator in this field of application, for example, typically consists of 6 stages. A high-voltage mains is used to charge the individual stages/capacitors with an individual capacity of 140 nF to 50 kV. The high-voltage pulse with an amplitude of 6×50 kV=300 kV and an aperiodic pulse length of approx. 1.5 μs for an adapted load of approx. 20 Ohm is generated during the process of discharging/conducting through. In the simplest case, for example if only one Marx generator is used, the contactors/spark gaps in the Marx generator are triggered via automatic breakdown. When switching several Marx generators in a system, triggering devices must be used for purposely igniting the spark gaps.

To achieve the aperiodic limiting case in the reactor, the electrode surface must have a value which is computed for a planar electrolytic resistance, using the following formula:

$$\frac{1}{R} = \frac{L \cdot F}{d}$$

For example, if R=20Ω is used for the generator resistance and the must value of 0.26 S/m is used for the electrolytic conductivity L, then the surface is computed to be 0.01 m² as guiding value for a distance d=0.06 m. This corresponds to a square of 10 cm×10 cm. Dividing this area into quadrants leads to four electrode pairs with an electrode surface of respectively 25 cm². In reality, the area would have to be formulated smaller since higher currents always flow along the inhomogeneous edge fields.

TABLE 1
Cell poration for the red-wine production (late-vintage burgundy red wine).

| | unfermented juice/young wine | | | | | | | |
|---|---|---|---|---|---|---|---|---|
| | (pre-clarified) | | | | | | | |
| | weight un- | | | | | wine | | |
| | fermented | centrifuge | | total | | | total | added |
| | juice | sludge | tannins | acid | pH | alcohol | extract | extract |
| | (° Oe) | (%) | (g/l) | (g/l) | value | (g/l) | (g/l) | (g/l) |
| control (ME) | 96.5 | 1.21 | 2.8 | 8.3 | 3.5 | 98.5 | 25.1 | 23.8 |
| product | | | | | | | | |
| cell-poration | 96.0 | 1.37 | 2.3 | 6.9 | 3.5 | 104.2 | 24.7 | 23.2 |
| product | | | | | | | | |

| | wine | | | | | | | |
|---|---|---|---|---|---|---|---|---|
| | total | | free | total | | | | |
| | acid | pH | SO₂ | SO₂ | tannins | color | color | ranking |
| | (g/l) | value | (mg/l) | (mg/l) | (mg/l) | intensity | nuance | number |
| control (ME) | 4.7 | 3.7 | 48 | 131 | 2.1 | 2.47 | 0.95 | 2.17 |
| product | | | | | | | | |
| cell-poration | 4.1 | 3.7 | 51 | 121 | 2.0 | 2.33 | 1.02 | 2.15 |
| product | | | | | | | | |

TABLE 2
Cell-poration for the white-wine production (riesling)

| | unfermented juice/young wine | | | | | | | |
|---|---|---|---|---|---|---|---|---|
| | weight un- | | | | | wine | | |
| | fermented | total | centrifuge | | | | total | added |
| | juice | acid | sludge | tannins | ferment. | alcohol | extract | extract |
| | (° Oe) | (g/l) | (%) | (g/l) | N-value | (g/l) | (g/l) | (g/l) |
| comparison product | 82 | 11.1 | 0.80 | 0.22 | 25 | 99.0 | 21.5 | 18.2 |
| (GTP) | | | | | | | | |
| control product | 77 | 9.2 | 0.97 | 0.33 | 32 | 96.2 | 19.4 | 19.3 |
| (only pumped) | | | | | | | | |
| cell poration | 79 | 8.6 | 0.80 | 0.57 | 37 | 98.9 | 20.6 | 20.5 |
| product (pumped) | | | | | | | | |

| | wine | | | | | | |
|---|---|---|---|---|---|---|---|
| | total | | free | total | | | |
| | acid | pH | SO₂ | SO₂ | tannins | potassium | ranking |
| | (g/l) | value | (mg/l) | (mg/l) | (g/l) | (mg/l) | number |
| comparison product | 6.7 | 3.1 | 44 | 85 | 0.26 | 498 | 2.3 |
| (GTP) | | | | | | | |
| control product | 6.7 | 3.1 | 43 | 83 | 0.33 | 585 | 2.5 |
| (only pumped) | | | | | | | |
| cell poration | 6.8 | 3.2 | 41 | 92 | 0.38 | 776 | 1.3 |
| product (pumped) | | | | | | | |

1. A process for an improved and gentler release of quality-enhancing constituents from grapes:
said process comprising the following steps:
A must obtained from red grapes and/or white grapes is heated to/held at a temperature that is above the freezing point for the must and can be adjusted up to temperatures that are standard for the traditional must heating;
Prior to the pressing operation for producing the young wine, the must is pumped/flows through a device/section of a device and is subjected therein to pulsed electrical fields for the irreversible opening of the walls of the biological cells in the grape skin, the process of electroporation, wherein the pulse duration is in the range of 0.5-3 ys;
During the electroporation, the must is subjected to such high-intensity pulsed electrical fields that on the one hand the grape-skin cells with a size of 7 to 10 μm experience a potential difference of at least 100 volt while, on the other hand, the streamer-formation limit of approximately 1000 kV/cm is not reached;
The specific energy dose for the electroporation of cells is adjusted to between 10 and 40 kJ per kilogram of must, at a must temperature ranging from 10° C. to approx. 40° C., and is adjusted upward by a factor of 2 to 4 times for a temperature below approx. 10° C. and adjusted downward by a factor of 2 for a temperature above approximately 40° C.; Following the electroporation process, the must undergoes a predetermined reactor residence time for releasing the quality-enhancing constituents from the fruit skin into the must, wherein this release is monitored by taking at least one must sample;
The residence time is followed by a pressing operation for producing the young wine.

2. The process according to claim 1, characterized in that following the electroporation the white-grape must is subjected to a reactor residence time lasting from several minutes to several hours.

3. The process according to claim 1, characterized in that the reactor residence time for the red-grape must ranges from approximately 1 hour to several days, following the electroporation.

4. The process according to claim 3, characterized in that the must for producing the young wine is at most subjected to a pressure that equals the pressure traditionally used for this process.

5. The process according to claim 4, characterized in that the must is moved continuously or in batches through the device/the section of the device used for the electroporation.

6. A young wine produced from grapes, as well as the wine produced therewith, characterized in that for producing this young wine and subsequently the table-ready wine, the must is subjected at least in part to the process step of electroporation as disclosed in claim 5.

7. A device for carrying out the electroporation of a grape must, characterized in that
The device/the section of the device for electroporation comprises a dielectric pipe, which represents the flow channel for the must and has a simple round or simple polygonal, but at least four-cornered cross section, wherein at least two spaced-apart electrodes are installed in the pipe wall for generating a pulsed, electrical field between the electrodes;
The electrodes are installed countersunk or flush with the wall or such that they project into the flow channel;
The complete surface area of the blank electrode surfaces projecting into the flow channel functions to control/delimit the flow between the electrodes,
wherein the clear cross section and the length of the flow channel for the device are designed such that the must as electrolytic load of the device has an electrical resistance which at most equals the impedance of a high-voltage pulse generator connected to the device.

8. The device according to claim 7, characterized in that the electrodes are positioned opposite each other, at an angle/offset to the flow axis, or in pairs perpendicular to the flow axis, and that in the case of more than two electrodes, these are oriented toward the flow axis or are positioned sequentially and spaced apart around the flow axis.

9. The device according to claim 7, characterized in that the electrodes are ring-shaped and follow successively, coaxial to the flow axis.

10. The device according to claim 7, characterized in that the electrodes are pin-shaped and follow each other along the flow axis, while spaced apart, or are positioned in a row in rotational direction around the flow axis.

11. The device according to claim 8, characterized in that the front of the respective electrode, which is exposed to the flow channel, has a rounded contour, such that with a pulsed field generation, at most an electrical field intensity of 1000 kV/cm can at any time be generated on all contour regions of the electrode and that the exposed electrode geometry does not initiate a backup in the flow of the must.

12. The device according to claim 8, characterized in that the flow channel and the therein installed electrodes are composed of an inert material that is suitable for processing food items, or that at least the surfaces coming in contact with the must are coated with this material.
